(19) United States
(12) Patent Application Publication
(10) Pub. No.: US 2012/0021906 A1
(43) Pub. Date: Jan. 26, 2012

(54) FUNGAL INOCULANT COMPOSITIONS

(76) Inventors: John Clifford Sutton, Ariss (CA); Todd Mason, Burlington (CA)

(21) Appl. No.: 13/259,757

(22) PCT Filed: Mar. 23, 2010
(86) PCT No.: PCT/CA2010/000429

§ 371 Date: Sep. 23, 2011

Related U.S. Application Data
(60) Provisional application No. 61/202,641, filed on Mar. 23, 2009.

(51) Int. Cl. A01N 63/04; A61P 43/00; A61K 9/14; A01P 21/00; A61K 35/66; C12N 1/14

(52) U.S. Cl. 504/117; 424/93.5; 435/254.1; 435/256.7; 435/256.5; 424/490

(57) ABSTRACT

An inoculant composition comprising fungal spores applied to a carrier having a moisture content of not more than about 5% is provided. A method of inoculating a plant to promote growth, enhance resistance to adverse conditions or promote re-growth is also provided comprising applying the inoculant composition to the plant.

### FIELD OF THE INVENTION

The present invention relates to fungal compositions, including fungal compositions useful as inoculants, as well as methods for producing and using such compositions.

### BACKGROUND OF THE INVENTION

The use of microbial inoculants to promote plant health is known. Generally, microbes, including bacteria and fungi, may be applied to a plant to improve plant nutrition, promote plant growth, provide resistance to disease and to treat disease. Examples of microbial inoculants include plant growth promoting rhizobacteria such as Rhizobium sp. which increase nitrogen nutrition in leguminous crops such as soybean and chickpeas, phosphate-solubilising bacteria such as Agrobacterium radiobacter, fungal inoculants including mycorrhizal fungi and endophytic fungi, such as Piriformis indica, which provide plant nutrition benefits, and composite inoculants which have shown synergistic effects on plant growth and nutrition.

In addition to their diverse utility, microbial inoculants can replace or significantly reduce the need to use harmful chemical fertilizers and pesticide treatments, which is becoming more important as regulations imposing stringent restrictions on the use of such chemicals come into force.

However, the preparation of some microbial inoculants, particularly fungal inoculants, is not without its challenges. For example, fungal spores are typically grown on a suitable substrate that is sterilized to prevent growth of contaminating bacteria and other microbes. Removal of the spores from the substrate to prepare a viable inoculant, such as by washing the substrate in water, generally risks germination and subsequent loss of activity of the spores, and initiates a very restrictive time limit within which the spores are useful as an inoculant. Accordingly, the spores are not normally removed from the substrate, but instead, the substrate bearing the fungus and its spores is ground up to form an inoculating composition in the form of a powder having a particle size that can appropriately be suspended in water and applied to a plant using standard techniques such as spraying. This grinding procedure is quite ineffective and inefficient, resulting in significant loss of spores (e.g. up to 90% or more) and a concomitant loss of spore activity in the final inoculant product.

There is, thus, a need to develop methods of preparing a fungal spore inoculant that improves upon currently used methods and improves upon the activity of the inoculant product.

### SUMMARY OF THE INVENTION

A novel inoculant composition has now been developed in which fungal spores are applied to a carrier that functions to stabilize the spores and thereby yield a non-germinating inoculant composition. The composition may be prepared employing a novel method of fungal spore recovery from a substrate to render a stable spore suspension comprising a spore concentration of at least about 1×10¹⁰ spores per mL.

Accordingly, in one aspect of the present invention, an inoculant composition is provided comprising fungal spores applied to a carrier having a moisture content of no more than about 5% to yield a stable non-germinating composition.

In another aspect, a method of preparing a fungal inoculant is provided comprising the step of applying a spore suspension to a carrier.

In another aspect of the invention, a stable fungal spore suspension is provided comprising a spore concentration of at least about 1×10¹⁰ spores per mL.

In another aspect of the invention, a method of preparing a stable fungal spore suspension is provided comprising:

1) inoculating a sterile substrate with a fungus and incubating under conditions suitable for fungal growth;

2) incubating the substrate under conditions suitable for fungal sporulation; and

3) removing the spores from the substrate by suspension in an aqueous solution and incubating the suspension to yield a spore concentration of at least about 1×10¹⁰ spores per mL.

In a further aspect of the invention, a method of inoculating a plant is provided comprising the steps of applying an inoculant composition to the plant, wherein the composition comprises fungal spores applied to a carrier having a moisture content of no more than about 5%.

These and other aspects of the invention are described by reference to the following description and examples.

### DETAILED DESCRIPTION OF THE INVENTION

An inoculant composition is provided comprising fungal spores adhered to carrier particles having a moisture content of not more than about 5%.

The term “fungal spores” is used herein to refer to spores of any fungus, particularly those which may beneficially be applied to plants to promote the growth, vigour and productivity thereof, to enhance resistance to disease, pests, and/or environmental stresses such as adverse weather or soil conditions, or to promote recovery of plants from injury and/or infection. Suitable fungal spores for inclusion in the present composition, include but are not limited to, spores of Clonostachys rosea that produce on asexual spores, such as strain 88-710, Trichoderma harzianum, Trichoderma koningii, Trichoderma (Gliocladium) virens, Paecilomyces lilacinus, Ulocladium atrum, Penicillium oxalicum and Penicillium bilai, and spores of non-pathogenic strains of Fusarium oxysporum.

To prepare a fungal inoculant according to an aspect of the invention, fungal spores are applied or adhered to carrier particles having a moisture content of not more than about 5%. The carrier functions to stabilize the spores in a dormant state and prevent germination thereof until the inoculant is used, e.g. to inoculate plants. Once the inoculant is exposed to water, the spores will germinate and colonize an appropriate host, e.g. the plant. Suitable carrier particles may have a particle size of less than about 0.5 mm, preferably less than about 0.4 mm, and more preferably less than about 0.35 mm. Examples of suitable carriers include, but are not limited to, skim milk powder; whey powder; whole milk powder; corn starch; potato starch; other starches; rice powder; dextrin; dextrose; finely milled seeds such as of barley, wheat, rye, and peas; finely ground corn cobs; finely ground distillers grain; chitosan; carboxymethylcellulose (CMC); finely ground peat (pH 6.0 or higher); finely ground coconut fibre; xanthan gum (e.g. extracellular polysaccharide of Xanthomonas campestris bacteria); talc; kaolin; bentonite; montmorillonite; very fine silicaceous or calcareous sand; Perlite™; and Turface™.

Additional components may be admixed with the carrier particles to facilitate preparation of the inoculant composition. For example, additives which assist in the preparation of a uniform inoculant composition may be combined with the carrier, for example, anti-clumping agents to prevent clumping of the carrier on addition of the spore suspension. Examples of anti-clumping agents include magnesium oxide, magnesium carbonate, or calcium carbonate. Such anti-clumping agents may be added to the carrier, e.g. in an amount of about 0.5 g to 1.0 g anti-clumping agent per kg carrier.

The inoculant composition is prepared by applying a suspension of fungal spores to a selected carrier. The spore suspension is prepared by admixture of spores in a sterile aqueous solution, such as water or buffer e.g. magnesium sulphate buffer at pH 7.0, at a concentration in the range of about 1-5×10⁹ spores/ml. The spores are substantially free from bacteria or contamination by other fungi. The spores may be prepared by growing the selected fungus on a sterile substrate, such as a sterile seeds (e.g. grains such as wheat, barley, etc.), and following a suitable amount of fungal growth, inducing spore formation under conditions that favour sporulation. As one of skill in the art will appreciate, sporulation conditions may vary depending on the selected fungus.

In one embodiment, a fungal spore suspension of C. rosea is prepared as follows. C. rosea is grown for several days on a substrate under conditions of high relative humidity (greater than 95%) and at a temperature in the range of 20-24° C. Sporulation is induced as the relative humidity is reduced over a period of time, e.g. a period in the range of about 10-20 days, in a controlled manner to about 20-25% and the moisture content of the substrate declines while the temperature is maintained. Spores are removed from the substrate and prepared as a suspension by admixture of the substrate with sterile water, shaking the mixture, filtering out clumped and coarse materials, gently centrifuging the filtrate, and resuspending pelleted material from centrifugation into a few ml of sterile water.

In this regard, it was surprisingly found that a highly concentrated fungal spore suspension was stable, e.g. the spores remained viable and active but did not germinate when maintained at 4° C. for an extended period of time. The stability of the spore suspension may vary with the concentration of spores in the suspension such that the greater the spore concentration, the greater the stability of the suspension and the longer the period within which the spores are non-germinating. In one embodiment, a suspension comprising a spore concentration of greater than about 1×10⁸ per mL, e.g. a spore concentration of about 1×10¹⁰ per mL, is stable for an extended period of at least about 2 weeks, and preferably for a period of greater than 2 weeks, e.g. 3 weeks, 4 weeks, 6 weeks or more, but readily germinated when subsequently incubated under favourable conditions for sporulation, such as on a standard agar medium at room temperature.

The spore suspension may be applied, for example as a spray, to a carrier while the carrier is churned, stirred, tumbled or shaken, or on the carrier in a fluid bed dryer, to form an inoculant composition. The volume of spore suspension applied to the carrier in the formation of the inoculant generally will not exceed 5% of the weight of the carrier, for example, about 50 mL of spore suspension may be applied to about 1 kg of carrier. The final concentration of spores on the carrier is generally about 1-4×10⁸ spores/gram of carrier.

The inoculant composition may comprise other additives to facilitate application or enhance inoculant performance. For example, the composition may include a dispersing agent such as acacia gum to facilitate application of the composition onto plant surfaces. Other suitable dispersing agent additives may include sodium stearate, Locust bean gum and vegetable oils such as soybean oil and corn oil.

The inoculant composition is in the form of a powder that may be applied as a dusting on plants or parts thereof including seeds. The inoculant may also be prepared for application by spraying by addition of water. Thus, in accordance with a method of the present invention, the fungal inoculant composition is applied to plants to promote growth, enhance resistance to disease or environmental stresses, or promote recovery from disease/stresses. Prior to application to a plant, the inoculant on the carrier (e.g. in the form of a powder) may be suspended in water, e.g. about 1 gram per liter water to provide the desired concentration of fungal spores for application to a given plant. As one of skill in the art will appreciate, the amount of inoculant used, e.g. concentration of spores, may vary from plant to plant. In one embodiment, the inoculant is prepared at a concentration of, for example, 10⁵ to 10⁶ spores per ml. In this regard, the inoculant may be spray applied to the entire plant, or any portion thereof, including the foliage and the roots. The inoculant may also be applied as a powder, i.e. without the addition of water, to the seeds or tubers of a plant. In this regard, the powder inoculant may comprise about 10⁷-10⁸ spores per gram of carrier. The powder inoculant may be applied to seeds at an amount of 1 gram of inoculant per kilogram of seeds.

Embodiments of the invention are described in the following specific example which is not to be construed as limiting.

### Example 1

### Preparation of Fungal Inoculant Using C. Rosea

Clonostachys rosea (asexual) was maintained in the long term as spores in 15% glycerol at −20° C. and −70° C. and in the short term on potato dextrose agar medium (PDA) as slants in culture tubes and in Petri dishes, all at refrigeration temperature (4° C.). Inoculum of Clonostachys rosea was produced in batches on barley or wheat seeds using the following protocol.

Sterilization of seeds. Seeds of any grain, such as wheat or barley (about 400 g in 400 mL water), were placed in clear plastic sterilization bags, such as #14 polypropylene breathable patch bags (48×20 cm). The opening of each bag was loosely sealed with tape. The bags were autoclaved for 1 hour at 121° C.

Production Clonostachys rosea spores. PDA in Petri dishes was inoculated with spores of C. rosea by placing a droplet of spore suspension containing 10⁶-10⁷ spores mL⁻¹ onto the medium in each dish and spreading the droplet over the agar surface with a cell spreader. The dispersed spores initiated numerous colonies which sporulated heavily at 22° C. and the spores were normally collected after 8 days. However, the plates with sporulating colonies may be kept at 4° C. for up to 1-2 months prior to use for inoculating seed.

Inoculation of the sterilized seeds. Spores were washed from the surface of the PDA in each Petri dish using 12 mL water containing about 0.04% Triton X-100 (or any suitable surfactant) and about 10 ml of the spore suspension was pipetted onto the seeds in each bag. Each bag was resealed with tape and shaken well to distribute the spores on the seeds. Relative humidity within the bags was about 95%.

Incubation of the inoculated seeds. In order to obtain abundant growth and spore production of Clonostachys rosea on the seeds without contamination of the seed culture by bacteria or other organisms, the bags were placed in a clean area in a temperature-controlled room at 20-25% relative humidity and 22-24° C. The bags were examined daily for white mycelial growth on the seeds. About every 3 days, each bag was shaken to redistribute the seeds, and mycelium on the seeds, and to maintain air passages among the seeds.

The spore production phase. Once a mass of mycelium had formed on the seeds, conditions were altered to enhance spore production. The colonized seeds were allowed to gradually dry (sporulation can be poor if high moisture persists). Progressive drying was achieved by placing the seeds into large translucent plastic boxes (e.g. 56 cm long×38 wide×15 cm deep) with lids. The inside of each box was surface sterilized by spraying with 70% alcohol and allowing the alcohol to dry. Colonized seed was placed in each box to form a loose layer several cm deep. The boxes with seeds were kept with the lids slightly open in a clean, well-ventilated room with a relative humidity of 20-25% and at a temperature of 20-24° C. The seeds were stirred and shaken every 4-5 days. Sporulation was generally heavy and the remains of the seed fairly dry (e.g. 20-30% moisture content) after about 1 week in the plastic boxes (e.g. about 24-30 days after the seeds were inoculated with spores).

Storage of seeds with sporulating Clonostachys rosea. At about 24-30 days following inoculation, the seeds with sporulating C. rosea were transferred to plastic sterilization bags with the necks closed and stored at 4° C. The “breathable” windows within the bags now provide sufficient aeration under these conditions. Clonostachys rosea can be stored on the seeds for several months at 4° C.

Recovery of spores from the colonized seeds. Sporulating seeds and sterilized water (containing 0.04% Triton X-100) were placed into a screw-capped jar and shaken vigorously for 1 minute to dislodge as many spores as possible into the water. About 1.8 L water was used to prepare a 1.5 L spore suspension because the colonized seeds soak up about 300 mL water. The seed residues were separated from the water suspension using any suitable apparatus, e.g. a centrifugal separator. The water suspension was then filtered first through a strainer (about 200 μm in size or larger) to remove any relatively large clumps, such as conidiophore clumps. Further filtering was then conducted in view of spore size (approximately 4-9 μm) and to remove smaller conidiophore clumps that are commonly 50-100 μM which can block fine sprayer nozzles. Filter sizes of 100 or 200 mesh are generally suitable. Filtering may be gravitational (vacuum not necessary but may speed up filtration). Filtration generally gives very “clean” spore suspensions (i.e. free from contaminating particles that are visible using standard light microscopes, including bacteria).

Following filtration, the spore suspension was concentrated by centrifugation at fairly low speed. For example, for a centrifuge accommodating six 250 mL plastic centrifuge bottles, 220 mL spore suspension was placed in each bottle and centrifuged at 3000 rpm for 5 minutes. The spore-containing pellet was re-suspended in about 20-25 mL sterile water plus surfactant. Spore concentration was about 2-5×10¹⁰ per mL. This spore suspension was stable to germination at 4° C. for up to at least about 14 days.

The number of spores per mL suspension was readily estimated by preparing serial dilutions of the spore suspensions in water and examining the diluted suspensions on a hemacytometer. Viable spores per mL spore suspension was determined by plating serial dilutions of the spore suspensions onto PDTSA (PDA containing Streptomycin antibiotic against many kinds of bacteria and Triton X-100 to limit rate of colony growth). Colonies were counted after 3-6 days and the counts were used to estimate densities of spores in the suspensions.

### Note on Spore Size:

Clonostachys rosea produces spores on two types of spore bearing branches (conidiophores) as follows:
1. Primary (verticillate) conidiophores.
   Spore size is relatively large: 7.6-9.0 μm long and 2.8-3.4 μm wide. Spores are often not curved and many lack a hilum (central indentation on one side like a seed of a white or black bean seed).
2. Secondary (penicillate) conidiophores.
   Spore size is smaller: 4.8-5.6 μm long and 2.4-3.0 μm wide. Spores are slightly curved and broadly rounded with one side slightly flattened with a hilum (bean like) and the other broadly rounded.

The size of some spores produced on the respective kinds of conidiophores may fall beyond the stated sizes.

### Note on Water Quality:

Sterile distilled water or sterile de-ionized water was used for production of inoculum and for preparing formulations (e.g. free from chlorine, other anti-fungal components and other microbes). Distilled water or de-ionized water was used for application of fungal inoculant onto plants.

Application of the spores onto a carrier material. For storage, distribution and use in crops the spores were applied to a suitable carrier material. Examples of carrier materials for spores of Clonostachys rosea include: skim milk powder; whey powder; whole milk powder; corn starch; potato starch; other starches; rice powder; dextrin; dextrose; finely milled seeds such as of cereals and legumes; finely ground corn cobs; finely ground distillers grain; chitosan; carboxymethylcellulose (CMC); finely ground peat (pH 6.0 or higher); finely ground coconut fibre; xanthan gum (=extracellular polysaccharide of Xanthomonas campestris bacteria); talc; kaolin; bentonite; montmorillonite; very fine silicaceous or calcareous sand; Perlite™; and Turface™.

The volume of spore suspension applied to the carrier (skim milk powder) was about 5% of the weight of the carrier. Example: maximum of 50 mL spore suspension per kg carrier. The spore suspension was applied to the carrier as a very fine spray while the carrier material was continuously churned, stirred, tumbled or shaken so as to achieve a highly uniform distribution of the spores on the carrier. If the concentration of spores in the suspension is 4×10⁹ per mL water and the final product should contain 2×10⁸ spores per gram of carrier, then 50 mL of the suspension was sprayed onto 1 kg of carrier. Since some spores may be lost during the application process, 6×10⁹ spores per mL, for example, may be applied to the carrier. In the event that the spore concentration is higher than desired, the mixture may be diluted appropriately with carrier (no spores on it).

To prevent clumping of the carrier on addition of the spore suspension, an anti-clumping agent such as magnesium oxide, magnesium carbonate, or calcium carbonate (0.5 g to 1.0 g anti-clumping agent per kg carrier) was added.

Yields. Yield of colonized seed with spore production from 1 kg fresh seeds (after autoclaving, inoculation and incubation) was 500 g of seeds that were heavily colonized by the fungus and sporulating abundantly, especially on the surface of the seeds. In summary, 100 kg of fresh original seed gives about 40 kg of seed with sporulating C. rosea. This was sufficient for at least 750 kg of inoculant in which the carrier was skim milk powder.

This methodology was employed using a number of asexual C. rosea strains, including strain 88-710.

### Example 2

### Preparation of Fungal Inoculant Using Trichoderm

The procedure described in Example 1 was utilized to prepare an inoculant using Trichoderma harzianum. Spores were obtained and used to inoculate sterilized seed, inoculated seed was incubated, spores recovered from the seed and applied to skim milk carrier as described. Similar yields of inoculant were obtained.

### Example 3

### Application of Fungal Inoculant to Plants

Fungal inoculant was prepared as described in Example 1. Mini rose cuttings were dipped in the inoculant, prepared by combining inoculant powder (about 1 g) with water (about 1 litre) to promote rooting, growth, and vigor. Following growth of the plants, the plants were trimmed and sprayed with the inoculant to control Botrytis disease and to promote vigor and flowering.

The effects of Clonostachvs rosea inoculant applied to miniature roses at various stages of production on estimated percent senescent and dead leaves, numbers of flowers, and plant quality index at 80 days after planting is set out in Table 1. Generally, treatment of plants with C. rosea inoculant mproved plant vigor, quality and productivity. Treatment of cuttings improved vigor at the first and second trimming. Plants were also more vigorous at the first trimming, and at second trimming when treated as cuttings. All treated plants exhibited better compactness and, in contrast to the controls, little or no specking, and only marginal discoloration or premature senescence of the leaves.

As set out in Table 1, the percent senescent or dead leaves at 80 days was reduced by 55-64% in plants treated once as cuttings, and was 73-80% lower in plants treated once at the first or second trimming, or as cuttings and again at one of the two times of trimming (Table 1). Few discolored or dead leaves were present on plants treated three times. Applications to fresh or planted cuttings in combination with sprays after the first or second trimming, or after both trimmings, increased counts of flower buds and open flowers (Table 1). All C. rosea treatments improved the quality index, however combined treatment of cuttings with one or two post-trimming sprays generally gave superior quality (Table 1). In this regard, improved plant form, greater visual appeal of the foliage associated with cuticular appearance and pigmentation patterns, and superior size, color quality, and freedom from imperfections in the flowers were observed. Severity of root dieback following foliar trimming was 5-15% in treated plants compared to 30-40% in the controls and plants that had not yet been treated. Clonostachys rosea frequently sporulated on leaf and stem tissues of treated plants, but infrequently on tissues of untreated plants. No pathogens or diseases were found on treated plants.

TABLE 1

| Production stages | Senescent and | | Number of flowers¹ | | | | Quality | |
|---|---|---|---|---|---|---|---|---|
| when treated | dead leaves (%) | | Buds | | Open | | index² | |
| Untreated (control) | 15.0 | a³ | 7.6 | c | 1.7 | c | 4 | c |
| Fresh cuttings (FC) | 5.4 | bc | 10.7 | bc | 3.3 | bc | 7 | b |
| Planted cuttings (PC) | 6.7 | bc | 9.3 | bc | 2.7 | c | 7 | b |
| First trimming (T1) | 4.0 | c | 11.0 | bc | 4.7 | ab | 8 | ab |
| Second trimming (T2) | 4.0 | c | 14.0 | ab | 3.3 | bc | 8 | ab |
| FC + T1 | 3.7 | c | 11.7 | b | 4.3 | abc | 8 | ab |
| FC + T2 | 3.0 | cd | 15.0 | a | 5.7 | ab | 9 | a |
| FC + T1 + T2 | 0.7 | d | 15.0 | a | 7.0 | a | 10 | a |
| PC + T1 | 3.7 | c | 15.3 | a | 4.0 | bc | 9 | a |
| PC + T2 | 3.0 | c | 14.7 | a | 6.0 | bc | 9 | a |
| PC + T1 + T2 | 1.0 | d | 17.0 | a | 6.7 | a | 10 | a |

¹Flowers per plant.
²Scale of 1 to 10, 1 = very poor, 10 = excellent.
³Values in a column followed by the same letter are not significantly different (P ≧ 0.05, PLSD test).

### Example 4

### Application of Fungal Innoculant to Seeds

Treatment of lentil seeds with 1 g powder inoculant (prepared as described in Example 1) per kg of seed prior to planting was found to increase % germination and % emergence in comparison with untreated seeds. Treatment may also promote the rate of emergence and rate of vegetative growth, enhance crop fitness and resistance to environmental and biological stresses and may substantially increase seed yields and quality of the lentils.

Plant growth response following treatment, including plant height (P-H cm), shoot fresh mass (F-mass) and shoot dry mass (D-mass) of the lentils at day 14 and day 28 after planting, is set out in Table 2.

TABLE 2

| | Day 14³ | | | Day 28³ | | |
|---|---|---|---|---|---|---|
| | P-H | F-mass | | P-H | | D-mass |
| Treatments | (cm) | (g)3 | D-mass (g)³ | (cm) | F-mass (g) | (g) |
| M¹ 0.00 g/kg | 17.9 | 0.54 | 0.05 | 32.6 | 3.37 | 0.64 |
| M 0.25 g/kg | 19.3 | 0.56 | 0.07 | 32.7 | 4.27 | 0.70 |
| M 0.50 g/kg | 19.6 | 0.56 | 0.07 | 33.1 | 4.27 | 0.73 |
| T² 0.00 g/kg | 16.1 | 0.27 | 0.04 | 26.7 | 1.37 | 0.21 |
| T 0.25 g/kg | 17.0 | 0.29 | 0.04 | 27.8 | 1.82 | 0.30 |
| T 0.50 g/kg | 17.2 | 0.28 | 0.04 | 28.2 | 1.94 | 0.33 |

¹M means seeds planted in Soil Mix LC1
²T means seeds planted in Top soil mixed with Perlite (95%:5% v/v)
³data Mean shoot fresh mass or shoot dry mass per plant.

Plant height. In the soil mix, inoculant at 0.25 and 0.5 g/kg seed, respectively, increased plant height by 7.8 and 8.6% at day 14 and by 0 and 1.5% at day 28. Respective values in the top soil were 5.6 and 6.8% at day 14, and 4.0 and 5.6% at day 28. The lower overall growth in the acid top soil compared to the soil mix should be considered in all comparisons such as of % increases in fresh and dry mass.

Shoot fresh mass: The inoculant treatments had a small effect (4-7% increase) on shoot fresh mass values by day 14 in the two soil types used. By day 28, treatment of the seed with 0.25 or 0.50 g Endophyte/kg each increased shoot fresh mass by 26.7% in plants grown in the soil mix. Overall growth was much less in the top soil (low pH) and numerous leaves fell from the plants (minor element deficiencies). Nonetheless, shoot fresh mass was increased by 32.9% at the 0.25 g rate and by 41.6% at the 0.50 g rate.

Shoot dry mass: After 14 days shoot dry mass at the 0.25 and 0.50 g rates was 40% greater than in the controls in the soil mix but no difference was seen in the top soil. After 28 days, the 0.25 and 0.50 g rates increased shoot dry mass by 9.4% and 14.1%, respectively, in the soil mix and by 43.1% and by 57.1%, respectively, in the top soil.

1. An inoculant composition comprising fungal spores applied to a carrier having a moisture content of no more than about 5%.

2. The composition of claim 1, wherein the carrier has a particle size of less than about 0.5 mm.

3-4. (canceled)

5. The composition of claim 1, wherein the carrier is selected from the group consisting of skim milk powder; whey powder; whole milk powder; starch; rice powder; dextrin; dextrose; finely milled seeds; finely ground corn cobs; finely ground distillers grain; chitosan; carboxymethylcellulose (CMC); finely ground peat (pH 6.0 or higher); finely ground coconut fibre; xanthan gum; talc; kaolin; bentonite; montmorillonite; silicaceous or calcareous sand; Perlite™; and Turface™.

6. The composition of claim 1, wherein the fungal spores are selected from the group consisting of spores of Clonostachys rosea, Trichoderma harzianum, Trichoderma koningii, Trichoderma (Gliocladium) virens, Paecilomyces lilacinus, Ulocladium atrum, Penicillium oxalicum, Penicillium bilai, and non-pathogenic strains of Fusarium oxysporum.

7. (canceled)

8. (canceled)

9. (canceled)

10. The composition of claim 1, comprising about 1-4×10⁸ spores/gram of carrier.

11. (canceled)

12. A stable fungal spore suspension comprising a spore concentration of at least about 1×10¹⁰ spores per mL.

13. (canceled)

14. A spore suspension as in claim 12, which is non-germinating for a period of at least about 2 weeks.

15. A method of preparing a stable fungal spore suspension as defined in claim 13 comprising:
1) inoculating a sterile substrate with a fungus and incubating under conditions suitable for fungal growth;
2) incubating the substrate under conditions suitable for fungal sporulation; and
3) removing the spores from the substrate by suspension in an aqueous solution and incubating the suspension to yield a spore concentration of at least about 1×10¹⁰ spores per mL.

16. The method of claim 15, wherein the sterile substrate is a seed.

17. The method of claim 15, wherein the inoculated substrate is incubated at a relative humidity of greater than 95% and at a temperature in the range of 20-24° C.

18. The method of claim 15, wherein sporulation is induced by reducing the relative humidity to about 20-25%.

19. A method of preparing a fungal inoculant as defined in claim 1, comprising the step of applying a spore suspension to the carrier.

20. The method of claim 19, wherein the spore suspension comprises a concentration in the range of about 1-5×10⁹ spores/ml.

21. The method of claim 19, wherein the volume of spore suspension applied to the carrier does not exceed 5% of the weight of the carrier.

22. The method of claim 21, wherein about 50 mL of spore suspension is applied to about 1 kg of carrier.

23. (canceled)

24. A method of inoculating a plant, any part of a plant or a seed, comprising the step of applying to the plant an inoculant composition as described in claim 1.

25. The method of claim 24, wherein the inoculant composition comprises about 10⁷-10⁸ spores per gram of carrier.

26. The method of claim 25, wherein the inoculant is applied to seeds at an amount of 1 gram of inoculant per kilogram of seeds.

27. The method of claim 24, wherein the composition is suspended in an aqueous solution comprising 10⁵ to 10⁶ spores per ml.

28. (canceled)

29. A fungal inoculant comprising fungal spores adhered to carrier particles, wherein the carrier particles stabilize the spores and prevent germination thereof.
